(19) United States
(12) Patent Application Publication
(10) Pub. No.: US 2009/0036458 A1
(43) Pub. Date: Feb. 5, 2009

(54) ENDOPARASITICIDAL TOPICAL COMPOSITIONS

(75) Inventors: Nahla Fattohi, Robbinsville, NJ (US); Moses Columbus Lawrence, Dayton, NJ (US); Shobhan Shashikant Sabnis, Pennington, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/182,253

(22) Filed: Jul. 30, 2008

Related U.S. Application Data
(60) Provisional application No. 60/962,754, filed on Jul. 31, 2007.

(51) Int. Cl. A61K 31/4985; A61P 31/00

(52) U.S. Cl. 514/250

(57) ABSTRACT

The present invention provides a composition which comprises: an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

Also provided is a method for the treatment of endoparasitic infection and infestation in a homeothermic animal.

### CROSS-REFERENCE TO RELATED APPLICATIONS

This application claims the benefit of U.S. Provisional Application No. 60/962,754, filed Jul. 31, 2007, the entire contents of which are incorporated herein by reference.

### BACKGROUND

Worms are the most common endoparasites of companion animals and infestations of worms are among the most critical of parasitic infestations in cats and dogs. Modern endoparasiticidal agents, such as moxidectin and praziquantel, have a wide margin of safety, considerable activity against immature or larval stages of parasites and a broad spectrum of activity. Nonetheless, the usefulness of any endoparasiticidal agent is limited by the inherent efficacy of the drug itself, its mechanism of action, its pharmacokinetic properties, features relating to the host animal, features relating to the target parasites and the form of administration.

In general, endoparasiticidal agents, such as praziquantel and a macrocyclic lactone such as moxidectin, for effective control are administered orally as a tablet or parenterally by a veterinarian. The “ideal” endoparasiticidal administrative form should have a broad spectrum of activity against mature and immature parasites, be easy to administer to companion animals, have a wide margin of safety, be compatible with other compounds, not require the assistance of a veterinarian and be economical. A further complication to the formulation of endoparasiticidal agents for use with companion animals is the cosmetic acceptability and non-irritability of the formulation when applied to the animal. Obviously, an acceptable formulation must be sufficiently easy to apply, dry within a reasonable period of time without impairment of the animal's appearance, be gentle on the animal's coat, non-irritating to the animal's skin and maintain its effectiveness on the animal through normal activities of the animal, such as exposure to sun and water. It must also be able to be applied to the animal in a small enough volume so that it can be applied so as to avoid the animal licking the area of application. Most desirably, the composition will provide the active ingredients in a formulation which will have at least a sufficient duration of activity, so as to avoid the necessity of frequent reapplication during this period of time. However, praziquantel is not highly soluble, and this characteristic has limited the development of veterinary compositions containing high concentrations of praziquantel.

Therefore, it is an object of this invention to provide a topical, endoparasiticidal veterinary composition containing (a) praziquantel and (b) a second endoparasiticidal agent selected from the group consisting of a macrocyclic lactone, imidacloprid or a combination thereof, which allows sufficiently high concentrations of each of the active ingredients and which is stable.

It is another object of the invention to provide a method for the prevention, treatment and control of endoparasiticidal infection or infestation in an animal, particularly a companion animal.

It is a further object of the invention to provide a broad spectrum gastro-intestinal worm treatment with simple stress-free topical application.

An additional feature of this invention is that the compositions provided offer high concentrations of active agents for maximum efficacy.

Other objects and features of the invention will be come more apparent from the detailed description set forth hereinbelow.

### SUMMARY

The present invention provides a composition which comprises: an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

Also provided is a method for the treatment of endoparasitic infection and infestation in a homeothermic animal.

### DETAILED DESCRIPTION

Many topical veterinary compositions require relatively high concentrations of active ingredients to ensure effective and long-lasting protection to the host animal and administration in sufficiently small volumes so as to avoid loss of the composition from run-off or licking by the animal. Typical “spot-on” applications of such compositions to the base of the neck of the animal aid in making the applied composition difficult for the animal to remove, but require that a relatively small volume be applied. However, the solubility of praziquantel frequently limits the abilitly to obtain high concentrations of praziquantel in such applications. Topical veterinary compositions containing praziquantel as one of the active ingredients are highly desirable due to the effective and persistent activity of praziquantel against a wide variety of intestinal worms.

Surprisingly, it has now been found that praziquantel and a macrocyclic lactone such as moxidectin may be formulated in a stable topical non-irritating composition by employing as a carrier 4-allyl-2-methoxyphenol. Accordingly, the present invention provides a topical veterinary endoparasiticidal composition which comprises: an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

Macrocyclic lactones are one chemical class of anthelmintics. As an example, “a macrocyclic lactone” may be an avermectin or a milbemycin or a combination thereof. Macrocyclic lactones, such as avermectins and milbemycins, are products, or chemical derivatives thereof, of soil microorganisms belonging to the genus Streptomyces. Such macrocyclic lactones are endoparasiticidal agents that are active against many immature nematodes and arthropods. Macrocyclic lactones suitable for use in the composition of the invention include: avermectins, such as abamectin, doramectin, ivermectin, selamectin or eprinomectin; and milbemycins, such as moxidectin or milbemycin oxime, or the like, preferably moxidectin.

The topical veterinary compositions of the present invention may include imidacloprid. Imidacloprid is a systemic insecticide.

The effective amounts of praziquantel and the macrocyclic lactone may be up to as high as 20% w/v of the total composition. For example, praziquantel may be present at about 10-15% w/v, preferably 10-12% w/v, and the macrocyclic lactone may be present at about 1.0-5.0% w/v, preferably 2.0-3.0% w/v. The effective amounts may vary according to the potency of the compounds, the method of application, the host animal, the target parasite, the degree of infestation, or the like. It is understood that effective amounts of less than 20% may be suitable for the composition of the invention. For example when the composition is administered in the form of a pour-on, spray or any topical administration suitable for use in large animals such as swine, sheep, horses or cattle, amounts of about 0.5-3.0% w/v, preferably 1.0-2.5% w/v, of praziquantel may be suitable and amounts of about 0.01-2.0% w/v, preferably 0.1-1.0% w/v, more preferably 0.5% w/v, of the macrocyclic lactone may be suitable.

As used herein, the term “w/w” designates weight/weight, “w/v” designates weight/volume, and the term “mg/kg” designates milligrams per kilogram of body weight.

In one embodiment, the 4-allyl-2-methoxyphenol carrier component is present in the topical veterinary compositions in an amount of about 20-70% w/v, preferably 25-65% w/v. In another embodiment, the 4-allyl-2-methoxyphenol carrier component is preferably present in the topical veterinary compositions in a minimum amount of about 20-30% w/v.

In addition to a carrier system consisting essentially of 4-allyl-2-methoxyphenol and the active agents, praziquantel and a macrocyclic lactone such as moxidectin, the topical composition of the invention may also include one or more additional ingredients. Examples of suitable additional ingredients include: stabilizers such as butylated hydroxytoluene, penetration enhancers such as polyglycolysed glycerides, e.g. LABRASOL™; anti-crystallizing agents such as polyvinylpyrrolidone (PVP); antioxidants; spreading agents, such as Crodamol PMP™; preservatives; adhesion promoters; viscosity modifiers such as polybutene polymers; UV blockers or absorbers; colourants; surface active agents, including anionic, cationic, non-ionic and ampholytic surface active agents; and those excipients conventionally employed in veterinary topical compositions. For example stabilizers, such as butylated hydroxytoluene, may be present in the composition of the invention in amounts of about 0-5% w/v, preferably about 0.1-1.0% w/v. Penetration enhancers such as polyglycolysed glycerides may be present in the inventive composition in amounts of about 0-40% w/v, preferably about 0-30% w/v. Anti-crystallizing agents such as polyvinylpyrrolidone may be present in the inventive composition in amounts of about 0-5%, preferably about 0.1-5.0%.

In one embodiment, the composition of the invention may further comprise a co-solvent such as γ-hexalactone or triethyl citrate. The co-solvent may be present in the composition of the invention in amounts of about 5.0-40% w/v, preferably about 15-30% w/v, more preferably about 20-26% w/v.

Excipients such as dyes, antimicrobial agents, antioxidants or mixtures thereof may be included in the composition of the invention. The amounts of said excipients suitable for use in the invention range from about 0-2.0% w/v.

Advantageously, the endoparasiticidal topical veterinary composition of the invention allows for high concentrations of the active ingredients and demonstrates no irritation to the skin/hide/hair of the host animal. Accordingly, the present invention provides a method for the treatment of an endoparasiticidal infection or infestation in a homeothermic animal, which comprises topically administering to said animal a composition which comprises an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

Examples of topical administrations suitable for use in the method of the invention include spot-on, pour-on, dip, wash, shampoo, foam, gel, lotion, or any of the conventional means of topically applying a liquid veterinary composition. The topical mode or administration will vary with the species and size of the host animal. As an example, for companion animals such as dogs or cats, a spot-on, gel, shampoo or wash, preferably a spot-on, may be suitable. For large agronomic animals such as cattle, horses or sheep, a pour-on or spray, preferably a pour-on, may be suitable.

Homeothermic animals suitable for treatment using the composition and method of the present invention include: swine, cattle, sheep, horses, goats, camels, water buffalos, donkeys, rabbits, fallow deer, reindeer, minks, chinchillas, raccoons, chicken, geese, turkeys, ducks, dogs, cats, or the like, preferably dogs, cats, swine, cattle, horses or sheep.

Endoparasitic infection or infestations suitable for treatment by the method of the invention include tapeworms, strongyles, Eencysted Cyathostomes, pinworms, hairworms, whipworms, ascarids, large-mouth stomach worms, bots or the like.

In actual practice, the composition of the invention may be administered in dose rates of mg of active ingredient per kg of body weight of the host animal. Dose rates suitable for use in the method of invention will vary depending upon the mode of administration, the species and health of the host animal, the target parasite, the degree of infection or infestation, the breeding habitat, the potency of the macrocyclic lactone, and the like. In general, amounts of said composition sufficient to provide about 8.0 mg/kg to 15.0 mg/kg, preferably about 10 mg/kg to 12 mg/kg of praziquantel per body weight of the animal and about 0.5 mg/kg to 3.5 mg/kg, preferably about 1.0 mg/kg to 2.5 mg/kg of a macrocyclic lactone such as moxidectin per body weight of the animal and are suitable.

Macrocyclic lactones suitable for use in the method of the invention include: avermectins such as abamectin, doramectin, ivermectin, selamectin or eprinomectin; milbemycins such as moxidectin or milbemycin oxime, or the like, preferably moxidectin.

For a more clear understanding of the invention, the following examples are set forth hereinbelow. These examples are merely illustrative and are not understood to limit the scope or underlying principles of the invention in any way. Indeed, various modifications of the invention, in addition to those shown and described herein, will become apparent to those skilled in the art from the examples set forth hereinbelow and the foregoing description. Such modifications are also intended to fall within the scope of the appended claims.

Unless otherwise designated, all parts are parts by weight. The term qs designates quantity sufficient to obtain a total of 100%.

### EXAMPLES

### Example 1

### Preparation of Endoparasiticidal Compositions

| Component | A | B | C | D |
|---|---|---|---|---|
| Description | % w/w | % w/w | % w/w | % w/w |
| Praziquantel | 10.50 | 10.53 | 11.80 | 11.9 |
| Moxidectin | 2.2 | 2.2 | 2.5 | 2.5 |
| BHT* | 0.40 | 0.45 | 0.50 | 0.50 |
| Crodamol PMPⁿ | — | 22.77 | — | — |
| LABRASOL ™** | 27.5 | 9.54 | — | — |
| Eugenol*** | 27.5 | 54.18 | 60.34 | 60.10 |
| γ-Hexalactone | qs | — | qs | — |
| Triethyl Citrate | — | — | — | qs |

*Butylated hydroxytoluene
**polyglycolysed glycerides
***4-allyl-2-methoxyphenol
ⁿPPG-2 Myristyl Ether Propionate

### Method of Preparation

The dry ingredients, such as moxidectin, praziquantel, BHT and PVP are mixed together and shaken until well dispersed. The resultant solid mixture is treated with the remaining liquid ingredients and stirred until a clear homogeneous solution is obtained.

### Example 2

### Preparation of Endoparasiticidal Compositions

Using essentially the same procedure described in Example 1 hereinabove, the compositions shown below are prepared.

| Component | A | B | C | D |
|---|---|---|---|---|
| Description | % w/v | % w/v | % w/v | % w/v |
| Praziquantel | 12.0 | 12.0 | 12.0 | 12.0 |
| Moxidectin | 2.50 | 2.50 | 2.50 | 2.5 |
| BHT* | 0.50 | 0.50 | 0.50 | 0.5 |
| Plasdone K- | — | — | — | 3.2 |
| 29/32** | | | | |
| LABRASOL ™*** | 29.0 | — | — | — |
| Eugenol**** | 30.0 | 65.0 | 65.0 | 65.0 |
| γ-Hexalactone | qs | qs | — | qs |
| Triethyl Citrate | — | — | qs | — |

*Butylated hydroxytoluene
**Polyvinylpyrrolidone (PVP)
***polyglycolysed glycerides
****4-allyl-2-methoxyphenol

### Example 3

### Evaluation of the Plasma Levels of Test Compositions

In this evaluation, test compositions prepared in Example 2 are administered to three dogs per treatment group by spotting the test composition between the front shoulders of the test animal at volumes providing a dose rate of 12 mg/kg of praziquantel and 2.5 mg/kg of moxidectin. Serum levels of moxidectin were determined at 3, 7 and 10 days after treatment (DAT). The results are shown in Table I. All test compositions showed acceptable appearance, when applied on dogs, 48 hours after treatment.

TABLE I
CANINE SERUM LEVELS OF MOXIDECTIN

| Body | | Dose | | | |
|---|---|---|---|---|---|
| Wt. | Test | mg/kg | Serum Moxidectin (ppb) | | |
| (kg) | Comp. | Prazi/Moxi | 3 DAT | 7 DAT | 10 DAT |
| 14.2 | 2A | 12.0/2.5 | 8.79 | 11.0 | 7.58 |
| 9.7 | 2A | 12.0/2.5 | 29.3 | 27.0 | 21.2 |
| 8.1 | 2A | 12.0/2.5 | 11.3 | 13.1 | 6.37 |
| 13.6 | 2B | 12.0/2.5 | 7.8 | 6.01 | 5.97 |
| 12.6 | 2B | 12.0/2.5 | 6.33 | 7.93 | 6.21 |
| 8.7 | 2B | 12.0/2.5 | 3.00 | 3.53 | 3.09 |
| 12.0 | 2C | 12.0/2.5 | 7.22 | 6.12 | 7.40 |
| 10.1 | 2C | 12.0/2.5 | 4.55 | 5.23 | 5.03 |
| 9.0 | 2C | 12.0/2.5 | 7.18 | 8.30 | 5.35 |

What is claimed is:

1. A topical veterinary composition which comprises: an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

2. The composition according to claim 1 wherein the macrocyclic lactone is selected from the group consisting of abamectin; doramectin; ivermectin; selamectin; eprinomectin;
moxidectin; and milbemycin oxime.

3. The composition according to claim 2 wherein the macrocyclic lactone is moxidectin.

4. The composition according to claim 1 wherein the effective amount of the macrocyclic lactone is about 1.0-5.0% w/v.

5. The composition according to claim 4 wherein the effective amount of the macrocyclic lactone is about 2.0-3.0% w/v.

6. The composition according to claim 1 wherein the effective amount of praziquantel is about 10-15% w/v.

7. The composition according to claim 3 wherein the effective amount of praziquantel is about 10-12% w/v.

8. The composition according to claim 1 wherein the 4-allyl-2-methoxyphenol carrier component is present in an amount of about 20-70% w/v.

9. The composition according to claim 1 wherein the 4-allyl-2-methoxyphenol carrier component is present in a minimum amount of about 20-30% w/v.

10. The composition according to claim 1 further comprising a penetration enhancer, a stabilizer and a co-solvent.

11. The composition according to claim 10 wherein the penetration enhancer is polyglycolysed glycerides and is present in the amount of about 0-40% w/v.

12. The composition according to claim 10 wherein the stabilizer is butylated hydroxytoluene and is present in the amount of about 0-5% w/v.

13. The composition according to claim 10 wherein the co-solvent is γ-hexalactone and is present in the amount of about 5.0-40% w/v.

14. The composition according to claim 13 wherein the penetration enhancer is polyglycolysed glycerides and is present in the amount of about 0-30% w/v and the stabilizer is butylated hydroxytoluene and is present in the amount of about 0.1-1.0% w/v.

15. The composition according to claim 14 wherein the effective amount is about 10-12% w/v of praziquantel and about 2.0-3.0% w/v of moxidectin.

16. A method for the treatment of endoparasiticidal infection or infestation in a homeothermic animal which comprises topically administering to said animal a composition which comprises: an effective amount of (a) praziquantel and (b) a second endoparasiticidal agent selected from the group consisting of a macrocyclic lactone, imidacloprid and a combination thereof; and (c) 4-allyl-2-methoxyphenol as carrier.

17. The method according to claim 16 wherein said composition is administered as a spot-on, pour-on, dip, wash, gel, shampoo, spray, foam, or lotion.

18. The method according to claim 16 wherein said animal is selected from the group consisting of dogs; cats; swine, cattle; horses; and sheep.

19. The method according to claim 16 wherein said endoparasiticidal infection or infestation is caused by tapeworms, strongyles, Eencysted Cyathostomes, pinworms, hairworms, whipworms, ascarids, large-mouth stomach worms or bots.

20. The method according to claim 16 having a composition wherein the macrocyclic lactone is moxidection.

21. The method according to claim 20 wherein said animal is a dog or cat.

22. A process for the preparation of a composition according to claim 1 which comprises admixing (a) praziquantel and (b) a second endoparasiticidal agent selected from the group consisting of a macrocyclic lactone, imidacloprid and a combination thereof to give a homogeneous dispersion; treating said dispersion with (c) 4-allyl-2-methoxyphenol to form a mixture; and stirring said mixture to obtain a clear solution.
